(19) United States
(12) Patent Application Publication
(10) Pub. No.: US 2024/0122740 A1
(43) Pub. Date: Apr. 18, 2024

(54) SOFT TISSUE SUSPENSION SYSTEM

(71) Applicant: timothy f. kelley, md inc., Newport beach, CA (US)

(72) Inventor: Timothy F Kelley, Newport Beach, CA (US)

(73) Assignee: timothy f. kelley, md inc., newport beach, CA (US)

(21) Appl. No.: 18/216,023

(22) Filed: Jun. 29, 2023

Related U.S. Application Data
(63) Continuation-in-part of application No. 17/648,955, filed on Jan. 26, 2022, now abandoned.

(51) Int. Cl. A61F 5/56; A61B 17/34

(52) U.S. Cl. CPC A61F 5/566; A61B 17/3468; A61B 2017/0464

(57) ABSTRACT

A method for anchoring soft tissue, such as a human tongue, to bone applied to minimizing snoring and sleep apnea in a human by properly positioning the tongue. A device is introduced through the patient's chin area through a drilled channel into the soft tissue of the tongue and then withdrawn in a manner whereby a portion of the device is secured to the patient's mandible by an engagement piece. A cord is utilized to apply tension to the engagement piece in a manner that allows for subsequent adjustment of tension on the engagement piece, both immediate and delayed.

### BACKGROUND OF THE INVENTION

### 1. Field of the Invention

A system for bone anchored soft tissue suspension applied to the human tongue and tongue base in a way that allows for normal function while preventing the tongue from becoming abnormally positioned, as in the airway during sleep, thus significantly reducing snoring and sleep apnea.

### 2. Description of the Prior Art

A number of techniques for reducing snoring and sleep apnea by correcting the abnormal tongue position in a human subject has been disclosed in the prior art. For example, U.S. Pat. No. 5,988,171 to Sohn et al discloses a technique for the treatment of human airway obstruction, sleep apnea and snoring by inserting a bone anchor into the mandible of the patient and fastening a suture to the bone anchor to suspend the tongue from the mandible. U.S. Pat. No. 8,973,582 to Rousseau discloses an implantable tissue suspension device and its method for use, the device including first and second filamentary elements bonded together only at a central connection region such that first and second leading and trailing legs of the respective first and second filamentary elements extend outwardly from the central connection region. The device is then implanted such that the central connection region extends laterally across a patient's tongue and the first and second leading and trailing legs of the first and second filamentary elements extend through the tongue such that ends thereof are positioned external to the genioglossus muscle. US Patent Application Publication No. 2012/031207 to Paraschac et al discloses at least one elongated body is sized and configured for implantation in a desired orientation in a tissue region in an airway. An array of projections extends from the elongated body and is resiliently coupled to the elongated body. The array of projections is in a normally biased outward extending condition sized and configured to engage tissue and resist a reorientation of the elongated body within the tissue region out of the desired orientation after implantation of the elongated body on the tissue region. The array of projections is sized and configured to resiliently yield by flexing inward against the elongated body during implantation of the elongated body in the tissue region. US Patent Application Publication No. 2014/0102460 to Catalano discloses a method for treating obstructive sleep apnea, the method providing a tethering device comprising an elastic filament having a distal end and a proximal end, and a flexible memory head mounted to the distal end of the elastic filament, advancing the tethering device through the tongue of a patient so that the flexible memory head of the tethering device is disposed against the back of the tongue, the elastic filament of the tethering device extending through the tongue, and securing the proximal end of the elastic filament to the mandible of the patient under tension, thereby restraining rearward movement of the tongue while the patient is sleeping.

### SUMMARY OF THE INVENTION

The present invention provides a method and apparatus for bone anchored soft tissue suspension applied to the treatment of snoring and sleep apnea by suspending the tongue and tongue base that allows for normal tongue function but prevents the tongue from becoming abnormally positioned in the patient's airway. This abnormal tongue positioning is a major contributing factor in snoring and sleep apnea.

The device of the present invention is composed of a multi prong, star shaped metal alloy (nitinal) or other rigid, flexible material acting as a soft tissue engagement piece that is attached to a thin cord of wire or suture that is placed through an introducing trocar. This multi prong, star shaped rigid, flexible soft tissue engagement piece is shaped like a “grappling” hook, with flexible prongs attached to a rigid central hollow shaft base. This multi prong flexible, rigid material is constructed such that the arms or prongs are able to be compressed down against the rigid central (hallow) shaft base creating a low profile such that it can be loaded into the central shaft of an introducing trocar. This multi prong rigid, flexible soft tissue engagement piece may also be used as a framework for attaching a synthetic “cloth like” material that, when opened, would be shaped like a “parachute” or “umbrella” or “flower with petals”, etc., thus increasing the surface area of tissue contact and tissue integration for improved suspension. The introducing trocar is a small-bore metal tube with a sharpened tip (needle), It serves in this system as a conduit through which the soft tissue engagement piece is guided to the proper position in the soft tissue, in this application, the tongue soft tissue. For this application of this device, the access point for the introducing trocar into the tongue soft tissue is through an introduction site adjacent to the genial tubercle of the mandible. This trocar introduction site is a bone channel that is formed by drilling a full-thickness channel through the lower central portion of the mandible at the symphysis from the outer cortex of the mandible through the inner cortex. This bone channel drill site at the mandibular symphysis is exposed through a small incision just below the front edge of the mandibular symphysis. This guided tissue suspension device introduction and deployment through the bone channel is controlled for accuracy by the premeasured length of the trocar and the skill and training of the operator. The introducing trocar is essentially a large, luer lock needle that serves initially as means to introduce local anesthesia as it is slowly inserted to the proper depth. Once properly positioned at a proper depth that is safely beneath the back surface of the tongue, the device is deployed through the introducing trocar into position in the tongue soft tissue by a deployment shaft that is slightly smaller than the bore of the introducing trocar. This deployment occurs when the deployment shaft with attached rigid, flexible tissue engagement piece and attached suture is advanced or pushed through the trocar into the tongue soft tissue. The device consists of the rigid, flexible soft tissue engagement piece temporarily attached to the deployment shaft with a suture firmly attached to the rigid, flexible soft tissue engagement piece. The suture runs through the hollow, central portion of the deployment shaft. The introducing tracer is then removed, leaving the deployment shaft temporarily still attached to the soft tissue engagement piece with attached suspension cord. Proper tension is then applied outward, away from the bone channel to the suspension cord to engage the multi prong rigid, flexible tissue engagement piece into the soft tissue. This serves to rotate the prongs outward from their resting place against the central shaft, similar in motion to opening an umbrella. This “locks” or engages the now open prongs into the soft tissue. Once properly engaged, the deployment shaft is disconnected and removed leaving the engaged multi prong rigid, flexible tissue engagement piece with attached cord suspended into the tongue soft tissue with the suspension cord exiting the bone channel introduction site. The suspension cord anchoring end is then securely attached to the mandible to apply constant tension. This constant tension allows for maintenance of tissue engagement and prevents tissue from migrating in the opposite direction. This, in turn, prevents the tongue from migrating into an abnormal position into the airway, thus preventing airway obstruction and/or snoring. The method for securing the attachment of the suspension cord to the mandible is variable and includes, but not limited to, a bone anchor device threaded onto the suspension cord and placed into the bone channel. The bone anchor device is designed to allow for tightening of the suspension cord up to 6-12 months after initial placement. An alternative attachment technique involves simply tying the suspension cord to itself, under proper tension, in front of the front side of the bone channel, creating a knot bigger than the diameter of the bone channel, resulting in the knot/cord complex being suspended against the front of the mandible. Another alternative deployment technique is similar to the one just described but involves deployment of two metal engagement piece/suspension cord devices that are tied together around a mandibular “bone bridge”, avoiding placement of any mandible anchoring hardware. This “bone bridge” is created by drilling two bone channels adjacent to each other, thus creating two separate introduction sites. Another variation employs the use of a barbed suture arranged in a way that allows it to be pulled through the drilled bone channel such that the barbs spring open once the desired length and tension is reached thus locking the suture in place against the front side of the bone channel. A further anchoring method into introduce a breakaway plug, or tapered plug, made of rigid material that is placed into the drilled bone channel to pinch the suspension cord into the bone channel. Another alternative method is to place a crimp on the suspension cord just in front of its entry point into the bone channel thus locking it in place. Another alternative technique involves introducing the device along the inferior (lower) border of the mandible and not through a drilled channel. This would still allow for anchoring to the rigid periosteal covering of the mandible in this location through the techniques described above.

It is believed that there is no device currently available that (1) suspends soft tissue through a bone channel introduction, deployment, and anchoring method/system, (2) applies the aforementioned bone channel introduction, deployment, and anchoring method/system to the tongue and tongue base, (3) uses a rigid, flexible (metal alloy) soft tissue engagement device that directly connects to and grabs, or firmly engages, the soft tissue for soft tissue suspension that is placed with a subsurface approach that avoids engaging the tongue on its surface, (4) provides a rigid, flexible alloy soft tissue engagement device acts a framework for synthetic material shaped in a way that when deployed, opens like an umbrella, parachute or flower, thus allowing for soft tissue suspension enhanced through synthetic material soft tissue integration; that allows for flexible bone anchoring without the use of separate bone anchoring hardware, and (5) that can be placed with the patient without general anesthesia.

The devices in existence are placed through incisions with a measure of soft tissue dissection that requires the patient to be placed under general anesthesia. The device of the present invention improves on this in that it can be placed through a local anesthesia approach, significantly reducing risk and minimizing soft tissue trauma. These devices rely on surface contact to the back of the tongue, thus exposing the device to contamination found on the tongue like saliva, food, mucus, etc. and hence, significantly increasing the risk of failure. The device of the present invention, on the other hand, is used under sterile conditions and relies on tissue suspension entirely through a subsurface approach and deployment, thus preventing the device from contacting any contaminated surfaces, including the airway, thus minimizing implant failure. In addition, these devices employ separate hardware for bone anchoring whereas the device of the present invention allows for anchoring through a pre-drilled bone channel without the need for separate hardware attached to the mandible or other bone anchor. The vector of suspension currently used is improved in that the vector of suspension for the device of the present invention is a direct, anterior-posterior vector, minimizing variability and direction of the suspension. The anterior anchor portion can be accessed and tightened up to twelve months after initial placement increasing the effective usefulness of the procedure and addressing the typical suspension slackening of prior suture suspension devices that rely entirely on simple suture soft tissue engagement. With simple suture soft tissue engagement, there is often a phenomenon of “cheese wiring” whereby the suture, under tension, slowly cuts through the soft tissue over time and with tissue movement, thereby slackening the tension on the soft tissue and reducing the suspension effect. The present invention system avoids this with its tightening feature and its more widely contacting and somewhat flexible metal alloy soft tissue engagement portion of the device and enhanced through the use of a composite metal alloy/synthetic material device with soft tissue integration.

### DESCRIPTION OF DRAWINGS

For a better understanding of the present invention as well as other objects and further features thereof, reference is made to the following description which is to be read in conjunction with the accompanying drawing wherein:

FIG. 1 illustrates the device of the present invention;

FIG. 2 illustrates the first step of the method of the present invention;

FIG. 3 illustrates the second step of the present invention;

FIG. 4 illustrates the full deployment of the engagement piece;

FIG. 5 illustrates the third step of the present invention;

FIG. 6 illustrates the fourth step of the present invention;

FIG. 7 illustrates the fifth step of the present invention;

FIG. 8 illustrates the sixth step of the present invention;

FIG. 9 illustrates the seventh step of the present invention;

FIG. 10 illustrates the eighth step of the present invention;

FIG. 11 illustrates the ninth step of the present invention; and

FIGS. 12-14 show the variations of the rigid, flexible soft tissue engagement piece.

### DESCRIPTION OF THE INVENTION

In accordance with the first step of the present invention, an incision is made in the chin at a location along the lower front edge at a position referred to as the submental crease. The soft tissue overlying the mandible in this area is then dissected off. A channel is then drilled through the mandible, the trocar/deployment shaft/metal tissue engagement piece/suture then being inserted along the lower edge of the mandible through rigid periosteum. The next series of steps are outlined and illustrated in the figures. Specifically,

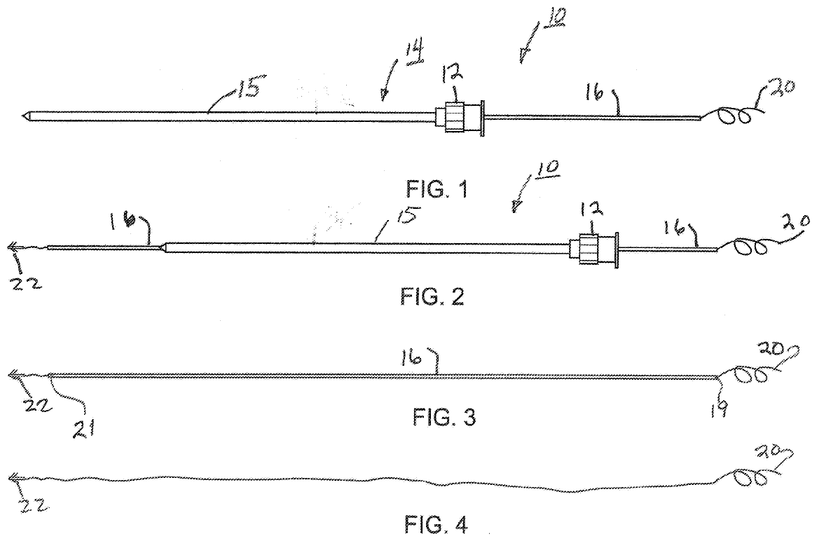

FIG. 1 illustrates the trocar 14 a luer lock fitting at the end of the trocar shaft 12, deployment shaft 16 exiting the luer lock end of the trocar, and the suture 20 exiting the end of the deployment shaft 16.

Looking right to left, FIG. 2 illustrates the deployment shaft 16 now exiting the front of the trocar with the rigid, flexible tissue engagement piece 22 at the leading edge of the deployment shaft.

FIG. 3 illustrates the deployment shaft 16 with the rigid, flexible tissue engagement piece 22 at the leading edge and the suture 20 passing through the deployment shaft 16,

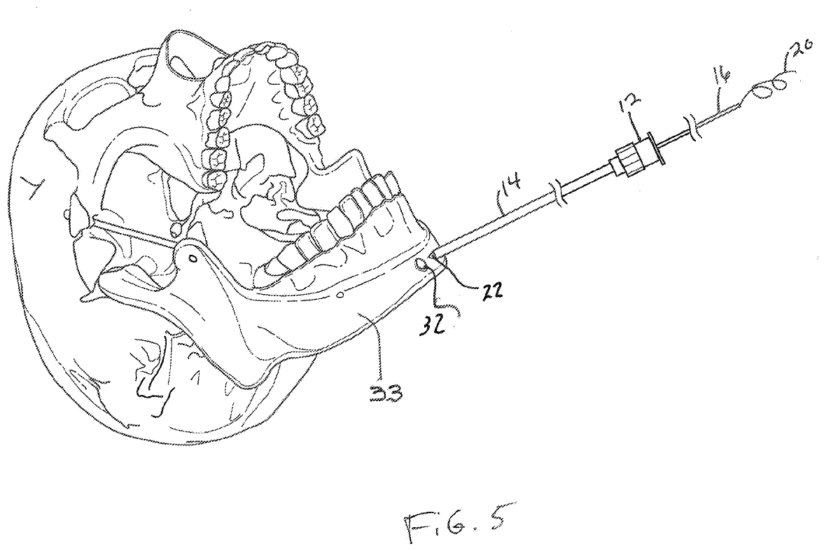

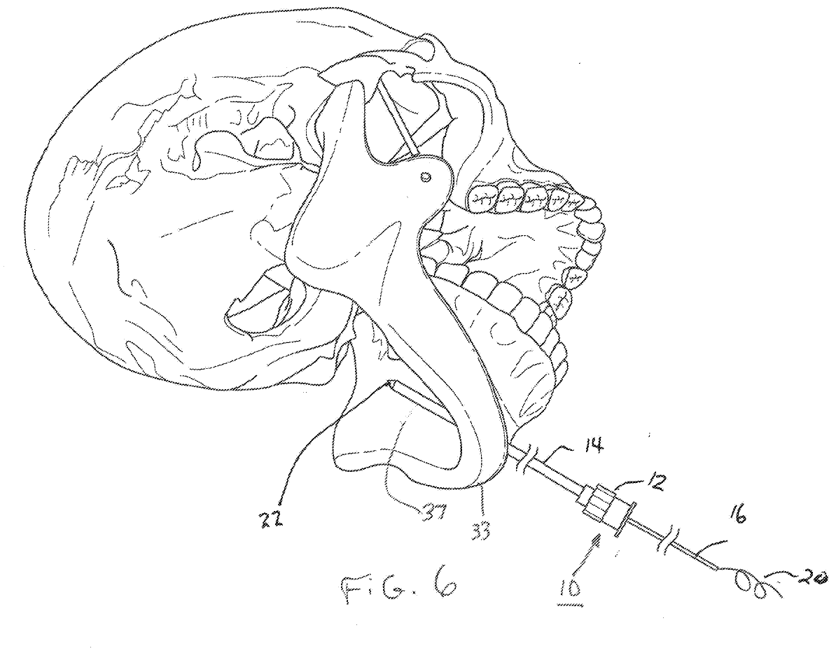

FIG. 4 illustrates the rigid, flexible tissue engagement piece 22 with attached suture 20.

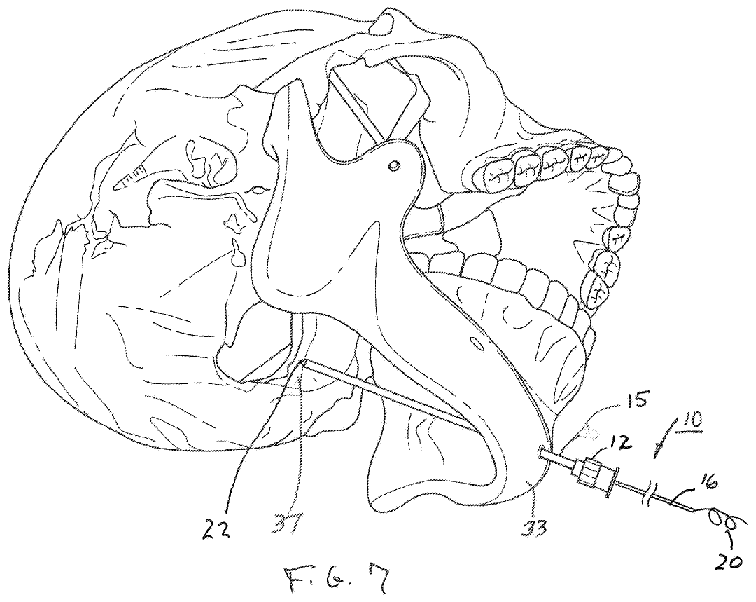

FIG. 5 illustrates the initial part of the deployment process with a bone channel 32 already drilled through the central portion of the mandible at the level of the genial tubercle;

FIG. 6 illustrates the trocar/deployment shaft/rigid, flexible tissue management piece/suture complex passing through the drilled bone channel in the mandible and into the soft tissue of the tongue at the predetermined position so as not to pass completely through the tongue.

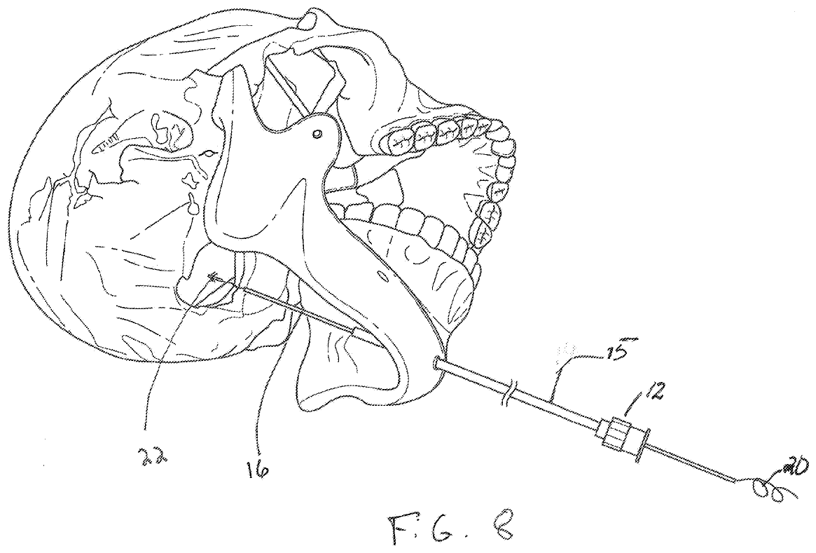

FIG. 7 illustrates the trocar/deployment shaft/rigid, flexible tissue engagement piece/suture at its predetermined position.

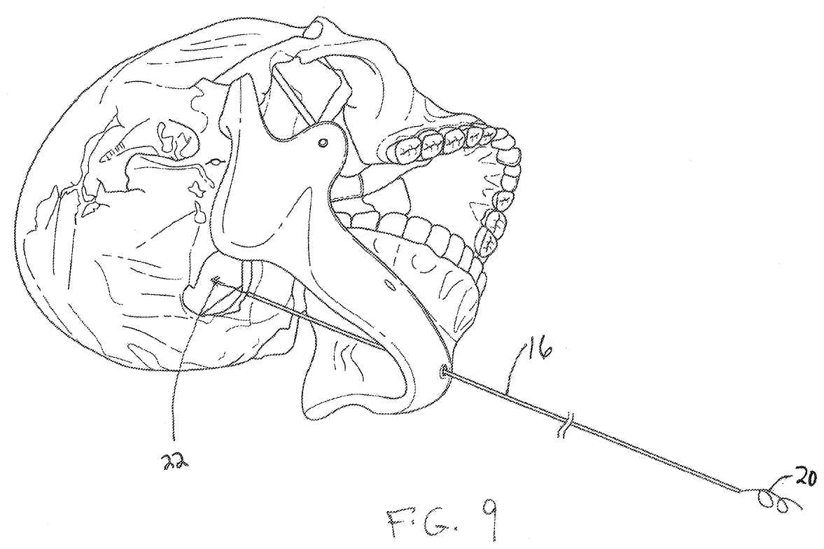

FIG. 8 illustrates the deployment shaft 16 with attached rigid, flexible tissue engagement piece now pushed through the trocar 14 with the rigid, flexible tissue engagement piece in the open position.

FIG. 9 illustrates the deployment shaft 16 with the attached rigid, flexible tissue engagement piece 22 after the trocar 14 has been removed.

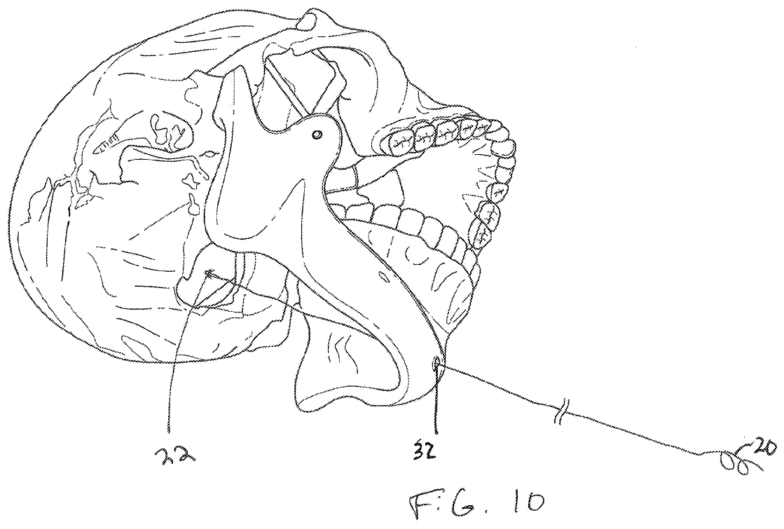

FIG. 10 illustrates the rigid, flexible tissue engagement piece 22 after the deployment shaft 16 has been detached and removed. The metal tissue engagement piece with suture 20 is now ready for final anchoring to the drilled bone channel 32 for full deployment.

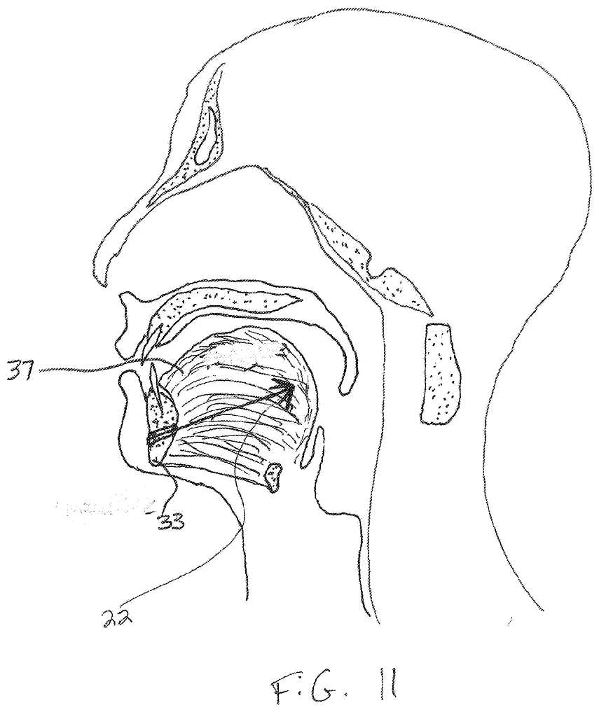

FIG. 11 illustrates the rigid, flexible tissue engagement piece 22 fully deployed below the back surface of the tongue and anchored to the mandible.

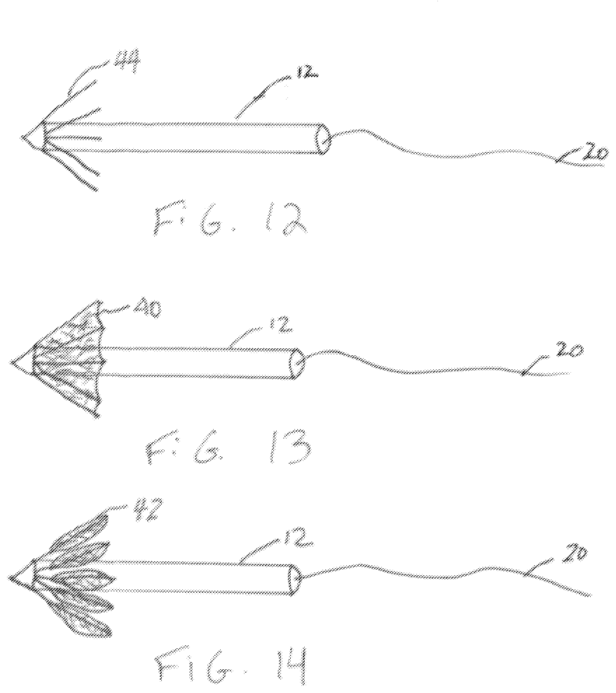

FIGS. 12-14 show variations of the rigid, flexible soft tissue engagement piece with the FIGS. 13 and 14 renditions including attached “cloth like” synthetic material secured to a rigid, flexible framework.

In summary, the device of the present invention is composed of a multi-prong metal alloy (nitinal) or other rigid, flexible material that functions as a soft tissue engagement piece that is attached to a thin wire cord, or suture, that is placed through an introducing trocar into soft tissue via a predrilled bone channel and then secured under tension. This rigid, flexible multi-prong soft tissue engagement piece serves as a framework for attached synthetic material in a variety of shapes and configurations as a composite to allow for improve soft tissue integration. This introducing trocar is small bore metal tube with a sharpened tip, similar to a large needle. It is guided to the proper position in the soft tissue, in this case the tongue soft tissue, through an introduction site at the symphysis of the mandible. This introduction site is a predriled bone channel at the symphysis of the mandible. Guided introduction into the proper position into the soft tissue is controlled for accuracy by the introducing trocar's premeasured length and the skill and training of the operator. Once properly positioned, the device is deployed from the introducing trocar into position in the tongue by a deployment shaft that is slightly smaller than the bore of the introducing trocar. The introducing trocar is then removed, leaving the metal engagement piece with attached suspension cord and the deployment shaft. Proper tension is then applied to the suspension cord and deployment shaft to engage the multi-prong tissue engagement piece. Once properly engaged, the deployment shaft is disconnected and removed. This leaves the engaged multi-prong rigid, flexible tissue engagement piece with attached cord in the tongue with the suspension cord anchoring end exiting the introduction site. The suspension cord anchoring end is then securely attached to the mandible with a bone anchor device or with other techniques described herein. This bone anchor device is designed to allow for tightening of the suspension cord 6-12 months after initial placement.

While the invention has been described with reference to its preferred embodiment, it will be understood by those skilled in the art that various changed may be made and equivalents may be substituted for elements thereof without departing from the true spirit and scope of the invention. In addition, many modifications may be made to adapt a particular situation or material to the teachings of the invention without departing from its essential teachings.

What is claimed is:

1. A method for applying a bone anchored soft tissue suspension to treat snoring and sleep apnea caused by the malposition of the tongue of a human, a human having a mandible bone anchor adjacent its chin area, comprising the steps of:
providing a trocar device;
introducing a rigid, flexible engagement piece that is attached to a thin cord, the engagement piece being positioned within a deployment shaft into said trocar;
guiding said trocar to a predetermined position within said soft tissue;
deploying said engagement piece from said trocar;
removing said trocar from the introduction site, leaving in place said engagement piece with the attached suspension cord and deployment shaft;
applying tension to said suspension cord thus engaging said tissue engagement piece; and
removing said deployment shaft, said multi prong metal piece with attached cord remaining in said soft tissue, one end of the extension cord exiting the introduction site and attaching said cord to the bone anchor.

2. The method of claim 1 wherein said soft tissue is the tongue of a human.

3. A surgical instrument for insertion into a human body cavity, said instrument having an opening extending through its length; and
an engagement piece attached to a thin, elongated member, said engagement piece with attached member being positioned within a deployment shaft, said deployment shaft positioned in said instrument opening.

4. The instrument of claim 3 wherein said engagement piece is capable of changing shape when tension is applied to said elongated member.

5. The instrument of claim 4 wherein said changing shape is in a form to attach to an adjacent object.

6. The instrument of claim 5 wherein said object is a bone within the mouth of said human body.

7. The method of claim 1 including the step of drilling a hole in the said mandible bone.

8. The method of claim 7 wherein said trocar is inserted through said drilled hole to said predetermined position.
